(19) United States
(12) Patent Application Publication
(10) Pub. No.: US 2008/0161337 A1
(43) Pub. Date: Jul. 3, 2008

(54) Use of Rifaximin for the Treatment of Chronic Prostatitis

(76) Inventor: Leonard Weinstock, St. Louis, MO (US)

Correspondence Address: HUSCH BLACKWELL SANDERS LLP, 190 CARONDELET PLAZA, SUITE 600, ST. LOUIS, MO, 63105-3441, US

(21) Appl. No.: 11/619,531

(22) Filed: Jan. 3, 2007

(51) Int. Cl. A61K 31/437; A61P 13/08

(52) U.S. Cl. 514/294

(57) ABSTRACT

Chronic prostatitis (CP) is a syndrome characterized by pelvic pain in one or more areas (perineum, lower abdominal, testicular and/or penile), urinary frequency, urinary urgency, sexual dysfunction and/or ejaculatory pain. CP type IIIa is the most common type of prostatitis and is defined by having sterile EPS and a leukocyte count greater than 1000/mm². A method of prevention of chronic prostatitis is disclosed that comprises orally administering a composition containing a therapeutically effective amount of rifaximin to a patient exhibiting symptoms of chronic prostatitis.

### CROSS REFERENCES

None.

### TECHNICAL FIELD OF THE INVENTION

The present invention relates generally to the medical field and, more particularly, to the use of rifaximin in the treatment of chronic prostatitis.

### BACKGROUND OF THE INVENTION

In general, rifaximin is well known as a non-systemic antibiotic (<0.4%) characterized by activity against a broad spectrum of enteric bacterial pathogens and the delivery of high concentrations of antibiotic to the gastrointestinal tract.

The antibiotic rifaximin was discovered in 1980 and originally patented in Italy as IT Patent 1154655 granted on Jan. 21, 1987. The related U.S. Pat. No. 4,341,785 to Marchi et al. discloses imidazo-rifamicyn derivatives having antibacterial utility, and the related process for preparing it. The '785 patent also discloses a pharmaceutical antibacterial composition and a method of using it to treat antibacterial diseases of the gastrointestinal tract. A further patent, U.S. Pat. No. 4,557,866 to Cannata et al. discloses a process for the synthesis of pyrido-imidazo rifamycins. The process is described as an improvement over the '785 patent to Marchi in that the later process provides unsatisfactory yields from an industrial point of view. The entire disclosures of the '785 and '866 patents are incorporated by reference herein.

Rifaximin is essentially a non-absorbable semi-synthetic antibiotic, related to rifamycin. The antimicrobial spectrum (in vitro) includes most gram-positive and gram-negative bacteria; and both aerobes and anaerobes.

It presents low risk for drug interactions (no effect on drugs metabolized by cytochrome p450 enzyme system) and about the same adverse properties as compared to a placebo. When ingested in tablet or pill form rifaximin is concentrated in the gastrointestinal tract and primarily excreted unchanged in the feces. It binds to the beta subunit of bacterial DNA-dependent RNA polymerase, which inhibits bacterial RNA synthesis. In contrast with other antibiotics, resistance to rifaximin is not plasmid-mediated but utilizes a chromosomal one-step alteration in the DNA-dependent RNA polymerase. In subjects using rifaximin no relevant resistance has been observed. Further, mutant resistant bacteria showed reduced viability and there is no systemic cross resistance for rifampin.

Since rifaximin is practically insoluble in water and is non absorbed (<0.4%) after oral administration, it can be used to treat localized diseases of the gastrointestinal tract. Rifaximin products specific for enteric pathogens of the gastro-intestinal tract are presently commercially marketed under various trade names—NORMIX® available from Alfa Wassermann S.p.A., Bologna, Italy; XIFAXAN® available from Salix Pharmaceutical, Raleigh, N.C.; REDACTIV® available from GlaxoSmithKline and FLONORM® from Schering-Plough. Since the solubility of rifaximin in water is approximately 1 .mu.gmL.sup.3 the drug is virtually undissolved when traveling through the GI tract. The relative insolubility of rifaximin is thought to influence bacterial susceptibility and subsequent eradication due to the invasive nature of some enteric pathogens (e.g., Salmonella and Campylobacter). The relative insolubility of rifaximin also leads to its negligible systemic absorption. Rifaximin has been known to be effective for treating infections that are localized to the gut and is not known to be suitable for treating systemic infections caused by invasive organisms.

Rifaximin has been marketed in Italy since 1985 under the trademark NORMIX® for treating acute and chronic intestinal infections from gram-positive and gram-negative bacteria and as adjuvant in the therapy of the hyperammonoaemia. At present NORMIX® is marketed in the shape of pharmaceutical compositions, orally administrable, made by tablets or by granulates containing suitable pharmaceutically acceptable excipients together with rifaximin, but also other pharmaceutical forms orally administrable like capsules, sugar coated tablets and syrups can be used. XIFAXAN® is marketed in the United States and Canada and includes rifaximin as the active ingredient. The formulation is used in the treatment of travelers' diarrhea caused by the noninvasive strains of Escherichia coli. XIFAXAN® is a non absorbable antiobiotic for gastrointestinal infections. Products similar to NORMIX® and XIFAXAN® are marketed in Mexico under the tradenames REDACTIV® and FLONORM®.

Other uses of rifaximin are disclosed in the following patents:

U.S. Pat. No. 5,886,002 to Ferrieri et al. describes use of rifaximin compositions in the treatment of diarrhea from cryptosporidiosis.

U.S. Pat. No. 5,352,679 to Ferrieri et al. describes use of rifaximin (INN) in formulations for treatment of gastric dyspepsia caused by Helicobacter pylori bacteria.

U.S. Pat. Nos. 5,314,904 and 6,140,355 both to Egidio et al. disclose compositions containing rifaximin for treatment of vaginal infections.

Known therapeutic uses of rifaximin, administered in a tablet form, include Clostridum difficile-associated diarrhea, Crohn's disease, Diverticular disease, Hepatic encephalopathy, Helicobacter pylori eradication, infectious diarrhea, irritable bowel syndrome, pouchitis, prophylaxis for GI surgery, small bowel overgrowth, traveler's diarrhea and ulcerative colitis. These therapies are directed to pediatric, adult and elderly subjects.

The use of Rifaximin in the treatment of chronic prostatitis has never been investigated.

Chronic prostatitis (CP) is a syndrome characterized by pelvic pain in one or more areas (perineum, lower abdominal, testicular and/or penile), urinary frequency, urinary urgency, sexual dysfunction and/or ejaculatory pain. CP type IIIa is the most common type of prostatitis and is defined by having sterile expressed prostate secretions (EPS) and a leukocyte count greater than 1000/mm². Leukocyte counts do not correlate well with symptoms. CP type IIIb is also a sterile prostate inflammatory condition, and patients with CP type IIIb have fewer white blood cells. The etiology for CP type III is unknown. Treatment with psychoanalysis, antidepressants, alpha-blockade, pentosan, balloon dilatation, microwave therapy, and prostate surgery are generally unsatisfactory approaches. The prevalence of CP and irritable bowel syndrome (IBS) ranges between 2-15% of men, however, coexistence studies of each are limited.

Current therapeutic alternatives for treatment of CP include change of long-term use of broad spectrum antibiotics, pentosan polysulfate sodium (heparin-like medication), antidepressants drugs and surgery. All of these treatments aim to provide symptomatic relief without providing a long-term cure. The effectiveness of these treatments varies. None of these treatments has been shown to be highly effective in a significant percentage of patients.

It would be desirable to provide an effective treatment for chronic prostatitis.

The present invention is directed to meeting one or more of the above-stated desirable objectives.

### SUMMARY OF THE INVENTION

The present invention provides a method of treating chronic prostatitis in a human comprising administration of rifaximin.

In general, in the method of the invention, a therapeutically effective amount of rifaximin is administered to a patient experiencing symptoms of CP. In a preferred embodiment, a dosage of approximately 550 mg per application is utilized. However, it is believed that dosages in the concentration range between about 400 mg and about 800 mg per application would also be effective. The duration of treatments with the invention formulations can be at daily, weekly or monthly intervals depending on the individual and the desired outcome. However, it is preferred that applications of rifaximin be administered three times daily for at least ten days.

In an alternate embodiment, a course of treatment with rifaximin is followed by long-term administration of the prokinetic medicine tegaserod.

Although this disclosure is directed to the preferred use of rifaximin, it is also within the scope of the invention that any non-systemic antibiotic can be included in the compositions and are included herein.

These and other objects, aspects, features and advantages of the present invention will become apparent from the following detailed description when taken in conjunction with the referenced drawings.

### BRIEF DESCRIPTION OF THE DRAWINGS

Not Applicable.

### DETAILED DESCRIPTION

Research suggests that small intestinal bacterial overgrowth (SIBO) may underlie some of the gastrointestinal and extraintestinal symptoms in patients with irritable bowel syndrome (IBS). Bacterial overgrowth with subsequent inflammation and neurochemical changes and visceral hypersensitivity suggest a possible role for SIBO in chronic prostatitis as well. Substance P has been evoked as having a role in neural hypersensitivity in each syndrome. Production of pro-inflammatory cytokines and reduced production of anti-inflammatory cytokines have been reported in IBS and theoretically could have a role in chronic prostatitis. Another potential theory for a relationship of IBS and chronic prostatitis relating to the similar findings of visceral hypersensitivity and elevated inflammatory mediators centers on the role of mast cells. Mast cells have been identified in a biopsy of the prostate in chronic prostatitis, and an increased number of mast cells have also been seen in the ileum of IBS patients. In IBS, mast cell degranulation, immune activation, and cytokine production may be triggered by bacterial endotoxins including lipopolysaccharides with or without bacterial translocation across the mucosal barrier. It is theorized in part that circulating inflammatory mediators may cause inflammation in the prostate and/or pelvic nerves as has been shown in IBS. Parenthetically, it has been suggested that the prostate tissue per se is not the source of the pain in chronic prostatitis.

In an open-label study, 78% of patients meeting the Rome I criteria for IBS had an abnormal lactulose breath test (LBT), a marker for the presence of SIBO. The role of SIBO in IBS was demonstrated when approximately 50% of patients improved to the point of no longer meeting the Rome criteria for IBS once their LBT was normalized by an antibiotic to demonstrate successful eradication of SIBO. The fact that extra-intestinal symptoms such as fatigue were improved if LBT became normal with treatment argues for the role of SIBO as a unifying explanation for the symptoms of these patients. In a subsequent, randomized, placebo-controlled study of 111 IBS patients, an abnormal LBT was found in 84% of patients with IBS. When patients were randomized to neomycin, a non-absorbable, gut-directed antibiotic, significantly better results were achieved than in those randomized to placebo. However, it was not simply being treated with antibiotics that mattered; it was whether the treatment of SIBO was successful. Specifically, when the LBT was normalized, patients reported 75% improvement of their symptoms. To further support the role of SIBO in IBS and functional bowel syndrome, a similar prevalence of 63% was shown in a study of 244 IBS patients undergoing the LBT. The effectiveness of antibiotic therapy with rifaximin and quinalone antibiotic therapy in reducing or eliminating IBS symptoms or reversing the breath test abnormality have now been reported in a number of studies. The clinical efficacy of antibiotics traditionally recommended for treating SIBO varies between 5% for metronidazole to 25% for neomycin and 40% for doxycycline. In contrast, the non-absorbed (<0.4%), gut-selective antibiotic rifaximin has been reported to be highly effective against SIBO. In a randomized, double-blind, parallel-group study of 21 patients with SIBO, rifaximin given for seven days at a dose of 1200 mg/day normalized the LBT in 70% of patients compared with 27% of patients treated with chlortetracycline. In this study improvement in functional gastrointestinal symptoms was greater with rifaximin than chlortetracycline. In a randomized, double-blind, parallel-group study of 34 patients SIBO and functional gastrointestinal symptoms, rifaximin improved LBT results and functional gastrointestinal symptoms. A recent randomized, double blind study of 86 IBS patients showed a statistically significant improvement in global IBS symptoms that was sustained for a period of ten weeks after a ten day course of rifaximin. The sustained improvement argues strongly that an antibiotic-sensitive mechanism such as SIBO is the underlying explanation for IBS. In further support of the long-term success of treatment of SIBO in IBS, a study of 82 IBS patients with abnormal LBT showed a combined global improvement rated great and moderate in 60% treated with rifaximin with a 54-day average follow up. In a parallel study of the role of SIBO in functional bowel syndrome, 20 patients with abnormal LBT showed a combined global improvement rated great and moderate in 63% treated with rifaximin with a 63-day average follow up.

Currently, patients with chronic prostatitis suffer disabling symptoms without the hope of finding treatment that can eliminate their symptoms. Physicians and patients find this world-wide disorder frustrating to manage. Prolonged treatment with quinalone and macrolide antibiotics was shown in one study to reduce EPS prostaglandin E 2 and beta-endorphin levels in patients with chronic prostatitis type III. This finding supports the theory that SIBO is a factor in chronic prostatitis since quinalone therapy has been shown to be effective in improving the breath test abnormality in SIBO. Furthermore there is no infection within the prostate per se, suggesting that the infective process may be outside of the prostate, such as in the gut.

Lactulose breath test to diagnose SIBO. In this test, the concentration of hydrogen in the exhaled breath before and after ingesting 10 g of lactulose, a non-digestible starch will be monitored by collecting exhaled breath sample every 20 minutes for 180 minutes. Diagnosis of SIBO will be based on the following criteria for a normal test: A clear hydrogen peak appearing 180 minutes after lactulose with a peak hydrogen concentration of less than 20 and greater than 5 parts per million (ppm). Any other pattern of gas excretion is generally considered abnormal. The breath samples may be analyzed on the QuinTron SC gas chromatography machine.

The role of SIBO in IBS and diseases other than chronic prostatitis, as well as the efficacy of breath tests in the diagnosis of SIBO is discussed in U.S. Pat. Nos. 6,861,053; 6,805,852; 7,056,686; and 7,048,906 to Lin and Pimentel, the disclosures of each of which are expressly incorporated by reference herein.

Principles and methodology of the lactulose breath test (LBT) to diagnose SIBO. In the absence of bacterial overgrowth, there is no hydrogen or methane in the breath. There is no source for hydrogen gas in humans other than bacterial metabolism of carbohydrates. When bacteria ferment carbohydrates or starches within the gastrointestinal tract, hydrogen and/or methane is released and then is absorbed through the mucosa. The gases are transported to the alveoli by the circulation and then the gases are released into the airways. In this commercial LBT, the concentration of hydrogen in the exhaled breath before and after ingesting 10 g of lactulose, a non-digestible starch, will be monitored by collecting exhaled breath sample every 20 min for 180 min. Lactulose powder (for example, KRISTALOSE™ produced by Mylan Bertak Pharmaceuticals, Inc, Research Triangle Park, N.C.) is mixed in 240 cc water. Samples are analyzed for hydrogen and methane by gas chromatography (for example, QUINTRON™ DP Plus manufactured by QuinTron Manufacturing, Milwaukee, Wis.). Diagnosis of SIBO are generally based on the following criteria for a normal test: A clear hydrogen peak appearing at or before 180 minutes after lactulose with a peak hydrogen concentration of less than 20 and greater than 5 parts per million (ppm). Any other pattern of gas excretion are generally considered abnormal.

There is no gold standard for determining the presence of bacterial overgrowth. The jejunal culture catheters do not pass beyond the proximal jejunum and are plagued by the problems of contamination by the mouth and upper GI tract and the difficulty of culturing bacteria (over 400 different bacteria are present in the GI tract and only 20% have been identified). Furthermore, bacterial overgrowth can be patchy. The breath tests provide indirect evidence of bacterial overgrowth. The sensitivity and specificity of the breath test for SIBO is dependant on the substrate used in the test. When glucose is the substrate (the GBT or glucose breath test) the sensitivity and specificity ranges between 60-90%. Lactulose is more sensitive but it is more specific since it is not absorbed from the gastrointestinal tract—the entire bacterial flora is sampled in this test. Glucose is absorbed in the first three feet of the small intestine and therefore it will not evaluate the presence of bacterial overgrowth from the mid jejunum to the ileum. Glucose underestimates the incidence of bacterial overgrowth by one half. This is reflected by the prevalence of abnormal GBT vs. LBT in IBS patients. It has been determined by older criteria that the LBT has a sensitivity of 68% and a specificity of 44%.

LBT test precision by the Quintron SC machine is excellent. This model is the most accurate machine commercially available. Furthermore, proper breath holding followed by immediate exhalation reduces variability in hydrogen level from 28% to 10%.

The potential interference from other ingested or endogenous compounds is not a concern in that patients can be directed to fast after a low carbohydrate dinner. Furthermore, having candy with sorbitol, Splenda and xylose, smoking, sleeping, and exercise can alter hydrogen excretion and these substances/activities should be forbidden before and during the test. Antibiotics and probiotics should be forbidden for four weeks prior to testing. Patients should brush their teeth in the morning although this has been recently tested and has no clinical testing effects.

The magnitude of differences between results at the two time points that is considered clinically and statistically significant is simply whether or not the test normalizes. This has been shown to be the key to determining clinical success in the treatment of patients with IBS.

Rifaximin. Rifaximin is a safe, nearly non-absorbable (<0.4%) antibiotic with action directed at the gut bacterial flora. Two properties of rifaximin provide excellent safety profile. The first property is that rifaximin requires bile salts for solubility. As bile salts are removed from the lumen in the terminal ileum, rifaximin becomes insoluble going across the ileocecal valve as it loses bile salts. The consequence of this site specific effect is that rifaximin spares the colonic flora and is therefore an antibiotic that exclusively targets the small intestine. The second property is that there is no plasmid transfer of resistance under rifaximin. Accordingly, there has been no clinically significant resistance reported in over 18 years of use in Europe under the brand name XIFAXAN®. Since rifaximin is nearly un-absorbable, reported side effects are rare and are likely to be explained by a change in host-gut bacterial relationship rather than true side effects of the medication. The following were reported with an incidence of ≧2% (XIFAXAN®: placebo): gas (11.3% vs. 19.7%), headaches (9.7% vs. 9.2%), abdominal pain (7.2% vs. 10.1%), rectal tenesmus (7.2% vs. 10.1%), defecation urgency (5.9% vs. 9.2%), nausea (5.3% vs. 8.3%), constipation (3.8% vs. 3.5%), fever (3.1% vs. 4.4%), vomiting (2.2% vs. 1.8%). These incidences as reported by XIFAXAN® treated subjects were not statistically different than that reported by placebo treated subjects. Vaginal yeast infection is not a listed adverse effect of this non-absorbable antibiotic. The following allergy or hypersensitivity events have been reported in post-marketing monitoring in Europe: allergic dermatitis, rash, angioedema, urticaria and pruritus.

An embodiment of the present invention provides a treatment for chronic prostatitis comprising oral administration of a therapeutically effective amount of rifaximin. A dosage of about 550 mg per application three times daily is indicated as therapeutically effective. However, it is believed that dosages in the concentration range between about 400 mg and about 800 mg per application would also be effective. This course of treatment is advantageously continued for a period of at least ten days. Rifaximin may be administered in a number of forms, which are disclosed and described in U.S. Pat. Nos. 4,341,785; 5,886,002; 5,352,679; 5,314,904; 6,140,355; and 7,045,620 and U.S. Publication No. 2006/0210592, the entire disclosures of which are expressly incorporated by reference herein.

In an alternate embodiment, a course of treatment with rifaximin is followed by long-term administration of the prokinetic medicine tegaserod (for example, ZELNORM® produced by Novartis Pharmaceuticals Corporation, East Hanover, N.J.) 3 mg nightly.

Other objects, features and advantages of the present invention will be apparent to those skilled in the art. While preferred medications, uses and steps of the method have been illustrated and described, this had been by way of illustration and the invention should not be limited except as required by the scope of the appended claims.

1. A method of treating chronic prostatitis comprising orally administering a composition containing a therapeutically effective amount of rifaximin to a patient exhibiting symptoms of chronic prostatitis.

2. The method set forth in claim 1, wherein said composition is a pharmaceutical composition.

3. The method set forth in claim 1, wherein said therapeutically effective amount of rifaximin is about 550 mg per application.

4. The method set forth in claim 1, wherein said therapeutically effective amount of rifaximin is in the range of about 400 mg to about 800 mg per application.

5. The method set forth in claim 3, wherein said method further comprises three applications of rifaximin daily for a period of at least ten days.

6. The method set forth in claim 1, wherein said composition is selected from tablets, capsules, sugar coated tablets or syrups.

7. The method set forth in claim 1, wherein said composition is a liquid preparation.

8. The method set forth in claim 1, wherein said composition is a dispersable or disintegrating tablet.

9. The method set forth in claim 1, further comprising the administration of a prokinetic medicine to said patient.

10. The method set forth in claim 9, wherein said prokinetic medicine is tegaserod.

11. The method set forth in claim 1, further comprising the step of conducting a lactulose breath test on said patient prior to administrating said therapeutically effective amount of rifaximin.

12. A method of treating chronic prostatitis, comprising the steps of:
conducting a lactulose breath test on a patient exhibiting symptoms of chronic prostatitis; and
orally administering a composition containing a therapeutically effective amount of rifaximin to said patient.
